(19) United States
(12) Patent Application Publication
(10) Pub. No.: US 2011/0312055 A1
(43) Pub. Date: Dec. 22, 2011

(54) METHOD OF PRODUCTION OF ALCOHOL

(75) Inventor: Karl Ragnar Weydahl, Bergen (NO)

(73) Assignee: Weyland AS, Radal (NO)

(21) Appl. No.: 13/122,296

(22) PCT Filed: Oct. 1, 2009
(86) PCT No.: PCT/GB09/02333

§ 371 Date: Aug. 16, 2011

(30) Foreign Application Priority Data
Oct. 2, 2008 (GB) 0818093.7

(51) Int. Cl. C12P 7/10; C13B 50/00; B01J 21/08; C01B 33/12; C12P 7/02; C12M 1/00; C13B 99/00; C01B 33/20

(52) U.S. Cl. 435/165; 127/9; 127/51; 423/326; 423/335; 435/155; 435/289.1; 502/232

(57) ABSTRACT

The invention provides a process for producing alcohol from a cellulosic material, said process comprising: hydrolyzing said cellulosic material with an aqueous acid to produce a hydrolysate; extracting acid and water from said hydrolysate with a water-miscible organic extraction solvent to yield (a) a first aqueous acidic solution containing said extraction solvent and (b) a residue containing sugars; subjecting said residue to an oligosaccharide cleavage reaction to yield an aqueous solution of fermentable sugars; fermenting said fermentable sugars and distilling alcohol from the resulting fermented mixture; contacting said first aqueous acidic solution with a pressurized fluid lipophilic solvent in non-gaseous form, which fluid solvent is substantially water-immiscible and is gaseous at ambient conditions, to yield a second aqueous acid solution and a solvent mixture of said extraction solvent and said fluid solvent; depressurizing said solvent mixture to release said extraction solvent for recycling; and, optionally, concentrating said second aqueous acid solution for recycling.

The invention relates to improvements in and relating to a process for the production of alcohol, especially ethanol or butanol, particularly ethanol, from cellulosic materials, in particular a process involving acid hydrolysis of cellulose.

Alcohol, produced by fermenting biomass, is rapidly becoming a major alternative to hydrocarbons such as natural gas and petroleum. While the current focus is on the production of ethanol from plant seed, e.g. maize, the magnitude of the demand for alcohol threatens a reduction in the land area devoted to food production and a desirable alternative to plant seed as the starting material is plant material other than seed, e.g. grass, wood, paper, maize husks, straw, etc. In this case the ethanol is produced by first breaking down the cellulose and hemicellulose (for convenience both are simply referred to as cellulose herein) into fermentable sugars. This may be done with enzymes but it is achieved most efficiently and economically by hydrolysis with strong acids, for example mineral acids such as sulphuric and hydrochloric acid. However for large scale commercial production of alcohol in this way, a major portion of the acid used must be recovered and recycled.

In WO 02/02826, the contents of which are hereby incorporated by reference, the inventors proposed such an ethanol production process in which the strong acid was recovered by contacting the hydrolysate with an organic extraction solvent, for example methyl ethyl ketone, with separation of the solid lignin and precipitated sugars to yield an acid solution comprising water, extraction solvent, acid and some dissolved sugars. The extraction solvent in the acid solution was then evaporated off under vacuum to be recycled and to leave an aqueous acid and sugar solution which was further evaporated off to yield a concentrated acid/sugar mixture, again for recycling.

The hydrolysate:extraction solvent ratio used in WO 02/02826 (see Example 1) is of the order of 3:8 and accordingly the energy requirement for recovery of the extraction solvent for recycling is a major portion of the overall energy demand for converting the cellulosic raw material into distilled ethanol.

We have now found that the extraction solvent recovery may be effected efficiently and with significantly lower energy demand by purging the acid/extraction solvent solution with a pressurized fluid lipophilic solvent in non-gaseous form which is gaseous at ambient conditions (e.g. 20° C. and 1 atmosphere pressure) and substantially immiscible with water.

Thus viewed from one aspect the invention provides a process for producing alcohol, particularly ethanol or butanol, especially ethanol, from a cellulosic material, said process comprising: hydrolyzing said cellulosic material with an aqueous acid to produce a hydrolysate; extracting acid and water from said hydrolysate with a water-miscible organic extraction solvent to yield (a) a first aqueous acidic solution containing said extraction solvent and (b) a residue containing sugars; subjecting said residue to an oligosaccharide cleavage reaction to yield an aqueous solution of fermentable sugars; fermenting said fermentable sugars and distilling alcohol (e.g. ethanol) from the resulting fermented mixture; contacting said first aqueous acidic solution with a pressurized fluid lipophilic solvent in non-gaseous form, which fluid solvent is substantially water-immiscible and is gaseous at ambient conditions, to yield a second aqueous acid solution and a solvent mixture of said extraction solvent and said fluid solvent; depressurizing said solvent mixture to release said extraction solvent for recycling; and, optionally, concentrating said second aqueous acid solution for recycling.

The pressurized lipophilic solvent is used in non-gaseous, i.e. liquid or supercritical, form. This solvent may for example be CO₂, a refrigerant gas (e.g. a halocarbon), a lower ether, or a lower alkane, alkene or alkyne, or mixture thereof. By lower in this regard is generally meant a carbon content of 1 to 4 atoms, especially 1, 2 or 3 atoms. Particularly desirably the solvent is a combustible material suitable for combustion to provide energy for one or more steps in the overall process. Especially preferably it is a material that is commercially available in liquefied form. Accordingly, the fluid solvent is desirably a liquefied hydrocarbon gas, e.g. liquefied petroleum gas (LPG) or liquefied natural gas.

The pressurized solvent may of course, like CO₂, have some solubility in water at ambient conditions; however, this should generally be no more than about 5 wt %, preferably no more than 1 wt %.

The pressurized solvent will preferably be contacted with the first aqueous acidic solution at a temperature between 0 and 80° C., especially 10 and 60° C., more particularly 15 and 50° C. The pressure used will be one sufficient to maintain the pressurized solvent in non-gaseous fluid form at the contact temperature used. If not already known, such pressures may readily be determined experimentally.

Depressurization will be to a pressure at which the pressurized solvent gasifies. If this is done at a temperature at which any significant part of the extraction solvent is gaseous, the depressurized material may be cooled to recover the extraction solvent in liquid form.

The overall alcohol production process may if desired be performed at a set of production sites, e.g. with production of the fermentable sugars on one site and fermentation and distillation at another. Equally, the acid hydrolysis, acid removal and extraction solvent removal may be performed at one site with the oligosaccharide cleavage and other downstream steps being performed at another site. Thus viewed from a further aspect the invention provides a process for the production of an aqueous solution of fermentable sugars from a cellulosic material, which process comprises: hydrolyzing said cellulosic material with an aqueous acid to produce a hydrolysate; extracting acid and water from said hydrolysate with a water-miscible organic extraction solvent to yield (a) a first aqueous acidic solution containing said extraction solvent and (b) a residue containing sugars; contacting said first aqueous acidic solution with a pressurized fluid lipophilic solvent in non-gaseous form, which fluid solvent is substantially water-immiscible and is gaseous at ambient conditions, to yield a second aqueous acid solution and a solvent mixture of said extraction solvent and said fluid solvent; depressurizing said solvent mixture to release said extraction solvent for recycling; and, optionally, concentrating said second aqueous acid solution for recycling. Viewed from another aspect the invention provides a process for the production of a sugar composition, said process comprising: hydrolyzing said cellulosic material with an aqueous acid to produce a hydrolysate; extracting acid and water from said hydrolysate with a water-miscible organic extraction solvent to yield (a) a first aqueous acidic solution containing said extraction solvent and (b) a residue containing sugars; drying said residue to yield said sugar composition; contacting said first aqueous acidic solution with a pressurized fluid lipophilic solvent in non-gaseous form, which fluid solvent is substantially water-immiscible and is gaseous at ambient conditions, to yield a second aqueous acid solution and a solvent mixture of said extraction solvent and said fluid solvent; depressurizing said solvent mixture to release said extraction solvent for recycling; and, optionally, concentrating said second aqueous acid solution for recycling.

The acid used in the process of the invention may be any strong acid, but will generally be an inorganic acid such as phosphoric or sulphuric acid. The use of sulphuric acid is preferred; the use of hydrochloric acid is generally not preferred. The use of a mixture of sulphuric and phosphoric acids, e.g. in a 1:1 to 4:1 volume ratio, especially about 2:1 volume ratio, is especially preferred.

The acid solution as contacted with the cellulosic starting material preferably corresponds to an acid:water weight ratio of 1:1 to 4:1, especially about 3:1. Acid solutions of the acid strengths conventionally used in strong acid hydrolysis of cellulosic materials may be used. It should be noted that acid and water may be added separately or that the initial acid added may be diluted or concentrated to yield the desired acid:water ratio.

The acid hydrolysis may be performed in conventional fashion. Typically, hydrolysis, which is exothermic, will be performed on a continuous basis, under cooling, e.g. water cooling, to maintain the hydrolysis mixture at 50 to 55° C. The acid solution:cellulosic material ratio is typically 2:1 to 4:1 by weight and the hydrolysis duration will generally be 1 to 4, especially about 2, hours. In this way the cellulose is broken down to produce oligosaccharides which can be precipitated out by the extraction solvent to yield a lignin/sugars slurry.

The extraction solvent used in the process of the invention may be any organic solvent which is capable of taking up water and acid and thereby causing the sugars to precipitate. Typically the solvent will be an alcohol, ether or ketone, e.g. having up to eight carbons. A mixture of such solvents, e.g. as described in WO 02/02826 may of course be used.

Contact between hydrolysate and extraction solvent is preferably effected in a counter flow column such that extraction solvent is added from below and removed from above and hydrolysate is added from above and the lignin/sugars slurry is removed from below. The slurry may be washed with extraction solvent if desired, it may be drained of liquids if desired, and it may be dried if desired. Alternatively it can be used directly for the oligosaccharide cleavage step after addition of water to bring the sugars into solution. The oligosaccharide cleavage reaction may be effected enzymatically or alternatively, and preferably, by acid hydrolysis. In practice the residue of acid retained in the unwashed slurry is adequate for oligosaccharide cleavage to proceed via such a second acid hydrolysis step. Alternatively further acid may be added, for example to bring the acid content of the sugar solution up to about 0.1 to 5 wt %, especially 0.5 to 2 wt %, particularly about 1 wt %. Addition of excess acid is undesirable as, following a second acid hydrolysis, the resulting hydrolysate must be neutralized to a pH suitable for the microorganisms responsible for fermentation (generally yeasts). This second hydrolysis may be effected under conventional conditions for weak acid hydrolysis of oligosaccharides, e.g. a temperature of 125 to 155° C., particularly about 140° C., a pressure of 2 to 7 bar, preferably 5-6 bar and a duration of about two hours.

Before fermentation, the fermentable sugars in aqueous solution are preferably filtered to recover any lignin. This is preferably washed to recover any entrained sugars for fermentation and compressed for use as a fuel, e.g. to provide energy for one or more of the steps in the overall alcohol production process.

Where the raw cellulosic material is rice straw. the lignin/sugars mixture will contain fine silica particles. These may be recovered by filtration, e.g. using differently sized meshes for lignin and silica or they may be recovered from the residue of the combustion of the lignin. Such silica particles are useful, e.g. as paint additives, pharmaceutical tabletting aids, or catalyst carriers (e.g. for olefin polymerization), and their collection and use form further aspects of the present invention. Viewed from a further aspect therefore the invention provides a process for producing particulate silicate comprising digesting rice straw with aqueous acid, precipitating sugars from the aqueous acid with a water-immiscible organic solvent, collecting the resulting mixture of precipitated sugars, lignin and particulate silica, and separating therefrom the particulate silica.

Viewed from still further aspects the invention provides a paint comprising silica particles isolated from rice straw; a pharmaceutical comprising silica particles isolated from rice straw as an excipient; and a particulate catalyst comprising silica particles isolated from rice straw and loaded with a catalyst. These products may be prepared by conventional means using other conventional ingredient.

The microorganism used in the fermentation step may be any microorganism capable of converting fermentable sugars to alcohol, e.g. brewer's yeast. Preferably however a yeast or yeast mixture is used which can transform the pentoses yielded by hemicellulose hydrolysis as well as the hexoses yielded by cellulose hydrolysis. Such yeasts are available commercially. The use of microorganisms that can transform pentoses to alcohol (e.g. Pichia stipitis, particularly P. stipitis CBS6054), particularly in combination with ones which can transform hexoses to alcohol, is especially preferred. Where fermentation is performed using microorganisms other than brewer's yeast (e.g. C. beijerinckii BA101), alcohols other than ethanol, in particular butanol, can be produced and these too can be used as biofuels. The invention covers the production of such other alcohols.

Distillation may be effected in conventional fashion.

The sugars produced using the invention can be fermented or respired by Baker's yeast or other microorganisms yeast to yield many different biological produced compounds such as glycerol, acetone, organic acids (e.g. butyric acid, lactic acid, acetic acid), hydrogen, methane, biopolymers, single cell protein (SCP), antibiotics and other pharmaceuticals. Specific proteins, enzymes or other compounds could also be extracted from cells grown on the sugars. The sugars moreover may be transformed into desired end products by chemical and physical rather than biological means, e.g. reflux boiling of xylose will yield furfural. The invention thus also covers the production of all such other produced compounds besides alcohols.

Viewed from another aspect, the invention provides apparatus for use in the processes of the invention, said apparatus comprising: a hydrolysis reactor; a first separator arranged to receive hydrolysate from said reactor and to discharge a sugar slurry; a second separator arranged to receive an extraction solvent/water mixture (i.e. containing acid) from said first separator and to discharge an aqueous acid solution; an acid reservoir arranged to supply acid to said reactor; an extraction solvent reservoir arranged to supply an organic extraction solvent to said first separator; a pressurized fluid reservoir arranged to supply a pressurized fluid lipophilic solvent in non-gaseous form, which fluid solvent is substantially water-immiscible and is gaseous at ambient conditions, to said second separator; and a depressurization chamber arranged to receive said lipophilic solvent (which will include extraction solvent) from said second separator and to discharge said lipophilic solvent in gaseous form.

The apparatus preferably also comprises components for recycling the acid and extraction solvent, and for feeding cellulosic material to the reactor. Conveniently, it also comprises components for the downstream handling of the sugar slurry, e.g. further hydrolysis reactors, reservoirs for a base for neutralizing the residual acid, fermentors and distillation units. To allow for continuous operation of the process when individual steps are performed batchwise, individual units within the apparatus may be duplicated, i.e. with such units being in parallel, so that one may be in operation while the other is being loaded/unloaded. This is particularly the case for the second acid hydrolysis, the fermentation, the distillation, and the lignin separation steps.

Where fermentation is performed using microorganisms other than brewer's yeast (e.g. C. beijerinckii BA101), alcohols other than ethanol, in particular butanol, can be produced and these too can be used as biofuels. The invention covers the production of such other alcohols.

Embodiments of the invention will now be described further with reference to the accompanying drawing, in which:

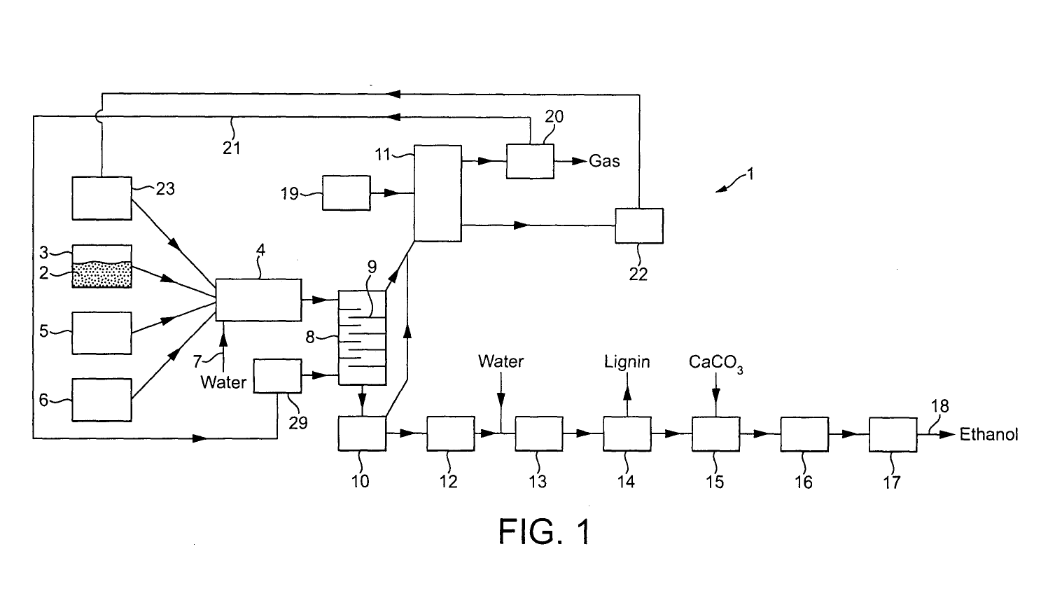

FIG. 1 is a schematic diagram of an apparatus according to the invention.

Referring to FIG. 1, there is shown an apparatus 1 for the conversion of wood pulp to ethanol. Wood pulp 2 is fed from hopper 3 into a hydrolysis reactor 4 containing a rotating screw operated to ensure a residence time for the wood pulp within the reactor of about two hours. The reactor is provided with a water-cooling jacket to maintain the hydrolysis mixture at about 50-55° C. Sulphuric and phosphoric acids and water, in a weight ratio of 2:1:1 are fed into reactor 4 from reservoirs 5 and 6, water feed line 7, and acid recycling reservoir 23. The hydrolysate from reactor 4 is fed to the top of a counterflow separation column 8 having internal plates 9 to delay through flow and so enhance separation efficiency. Into the base of column 8 is introduced an organic extraction solvent, methyl ethyl ketone from reservoir 29. Within the column 8, water and acid are taken up by the extraction solvent and lignin and precipitated sugars are passed from the base of the column to a continuous filtration unit 10. The acid/water/extraction solvent mixture is discharged from the top of column 8 and fed into a separator column 11.

The solid residue from filtration unit 10 is passed to a drier 12 and the dry lignin/sugar mixture is then dissolved in water and passed into a second hydrolysis reactor 13. The liquid from the filtration unit 10 is passed to separator column 11.

In the second reactor 13, a further acid hydrolysis is effected at 140° C. for two hours at 5-6 bar. The hydrolysate is filtered in filtration unit 14 to remove lignin (which is compressed and combusted to provide energy for the overall apparatus). The remaining solution of fermentable sugars is neutralized with calcium carbonate in neutralization unit 15 before being passed to fermentation unit 16 where brewers' yeast is added and fermentation is allowed to take place. The fermented mixture is then fed to distillation unit 17 where ethanol is distilled off via line 18.

The acid/water/extraction solvent in separator column 11 is purged under pressure with liquefied petroleum gas (LPG) from LPG reservoir 19. The LPG is led from separator column 11 to a depressurization chamber 20 where the pressure is reduced sufficiently to gasify the LPG and release the entrained extraction solvent as a liquid which is recycled via line 21 to reservoir 29. The remaining aqueous acid is fed from separator column 11 to evaporator unit 22 where water is removed under vacuum. The remaining acid, containing some dissolved sugar, is recycled to reservoir 23. The gasified LPG from depressurization chamber 20 is combusted to provide energy for the overall apparatus.

1. A process for producing alcohol from a cellulosic material, said process comprising: hydrolyzing said cellulosic material with an aqueous acid to produce a hydrolysate; extracting acid and water from said hydrolysate with a water-miscible organic extraction solvent to yield (a) a first aqueous acidic solution containing said extraction solvent and (b) a residue containing sugars; subjecting said residue to an oligosaccharide cleavage reaction to yield an aqueous solution of fermentable sugars; fermenting said fermentable sugars and distilling alcohol from the resulting fermented mixture; contacting said first aqueous acidic solution with a pressurized fluid lipophilic solvent in non-gaseous form, which fluid solvent is substantially water-immiscible and is gaseous at ambient conditions, to yield a second aqueous acid solution and a solvent mixture of said extraction solvent and said fluid solvent; depressurizing said solvent mixture to release said extraction solvent for recycling; and, optionally, concentrating said second aqueous acid solution for recycling.

2. A process as claimed in claim 1 wherein the alcohol produced is ethanol.

3. A process for the production of an aqueous solution of fermentable sugars from a cellulosic material, which process comprises: hydrolyzing said cellulosic material with an aqueous acid to produce a hydrolysate;
extracting acid and water from said hydrolysate with a water-miscible organic extraction solvent to yield (a) a first aqueous acidic solution containing said extraction solvent and (b) a residue containing sugars; contacting said first aqueous acidic solution with a pressurized fluid lipophilic solvent in non-gaseous form, which fluid solvent is substantially water-immiscible and is gaseous at ambient conditions, to yield a second aqueous acid solution and a solvent mixture of said extraction solvent and said fluid solvent; depressurizing said solvent mixture to release said extraction solvent for recycling; and, optionally, concentrating said second aqueous acid solution for recycling.

4. A process for the production of a sugar composition, said process comprising: hydrolyzing said cellulosic material with an aqueous acid to produce a hydrolysate; extracting acid and water from said hydrolysate with a water-miscible organic extraction solvent to yield (a) a first aqueous acidic solution containing said extraction solvent and (b) a residue containing sugars; drying said residue to yield said sugar composition; contacting said first aqueous acidic solution with a pressurized fluid lipophilic solvent in non-gaseous form, which fluid solvent is substantially water-immiscible and is gaseous at ambient conditions, to yield a second aqueous acid solution and a solvent mixture of said extraction solvent and said fluid solvent; depressurizing said solvent mixture to release said extraction solvent for recycling; and, optionally, concentrating said second aqueous acid solution for recycling.

5. A process as claimed in any one of claims 1 to 4, wherein said fluid solvent is liquefied petroleum gas.

6. A process as claimed in any one of claims 1 to 5 wherein a part of the said lipophilic fluid solvent released in gaseous for in the depressurisation step is combusted to generate energy. for use in the process and a further part of said lipophilic fluid solvent is condensed and recycled.

7. A process for producing particulate silicate comprising digesting rice straw with aqueous acid, precipitating sugars from the aqueous acid with a water-immiscible organic solvent, collecting the resulting mixture of precipitated sugars, lignin and particulate silica, and separating therefrom the particulate silica.

8. A paint comprising silica particles isolated from rice straw.

9. A pharmaceutical comprising silica particles isolated from rice straw as an excipient.

10. A particulate catalyst comprising silica particles isolated from rice straw and loaded with a catalyst.

11. Apparatus for use in a process as claimed in any one of claims 1 to 7, said apparatus comprising: a hydrolysis reactor; a first separator arranged to receive hydrolysate from said reactor and to discharge a sugar slurry; a second separator arranged to receive an extraction solvent/water mixture from said first separator and to discharge an aqueous acid solution; an acid reservoir arranged to supply acid to said reactor;
an extraction solvent reservoir arranged to supply an organic extraction solvent to said first separator; a pressurized fluid reservoir arranged to supply a pressurized fluid lipophilic solvent in non-gaseous form, which fluid solvent is substantially water-immiscible and is gaseous at ambient conditions, to said second separator; and a depressurization chamber arranged to receive said lipophilic solvent from said second separator and to discharge said lipophilic solvent in gaseous form.
